(19) United States
(12) Patent Application Publication
(10) Pub. No.: US 2024/0324943 A1
(43) Pub. Date: Oct. 3, 2024

(54) TREATMENT OF MENTAL HEALTH DIAGNOSES

(71) Applicant: Barbara Susan Schmitt, Windsor, CO (US)

(72) Inventor: Barbara Susan Schmitt, Windsor, CO (US)

(21) Appl. No.: 18/389,099

(22) Filed: Nov. 13, 2023

Related U.S. Application Data
(60) Provisional application No. 63/492,843, filed on Mar. 29, 2023.

(51) Int. Cl. A61B 5/378; A61B 5/00; A61B 5/16; G16H 20/10

(52) U.S. Cl. CPC A61B 5/378; A61B 5/165; A61B 5/7435; G16H 20/10

(57) ABSTRACT

Conventional methods of integration in psychedelic-assisted treatment utilize questions and scales that can be triggering to a patient who is recovering from trauma. Disclosed methods treat the patient as the medicine by utilizing a visual implement that uses visual content and creative expression to guide the patient towards improved outcomes, once the patient has egressed from the psychedelic experience. The visual implement may be embodied in a tablet computer, with a touch-panel display, that enables the patient to both view and create and/or outline the visual content. The tablet computer may execute a software application that enables a clinician to choose the appropriate visual content and/or strategy for engaging the patient.

### CROSS-REFERENCE TO RELATED APPLICATIONS

The present application claims priority to U.S. Provisional Patent Application No. 63/492,843, filed on Mar. 29, 2023, which is hereby incorporated herein by reference as if set forth in full.

### BACKGROUND

### Field of the Invention

The embodiments described herein are generally directed to the treatment of mental health diagnoses, and, more particularly, to the safe and effective integration of a patient (e.g., during a psychedelic-assisted treatment).

### Description of the Related Art

A growing body of research suggests that psychedelic substances can aid in the treatment of mental health diagnoses, including anxiety, depression, post-traumatic stress disorder (PTSD), and substance use disorder or addiction, which all share an underlying risk of suicidality. Classic examples of psychedelic substances include, without limitation, lysergic acid diethylamide (LSD), mescaline or peyote, psilocybin, ayahuasca, and N,N-Dimethyltryptamine (DMT). Non-classic examples of psychedelic substances include, without limitation, ketamine, ibogaine, 3,4-Methylenedioxymethamphetamine (MDMA), salvinorin A, and tetrahydrocannabinol (THC).

One example of psychedelic-assisted treatment is psychedelic therapy. Typically, psychedelic therapy involves a preparation stage, ingestion stage, and integration stage. During the preparation stage, the patient will meet with a clinician to ensure that the patient does not have any contraindications to the treatment, build rapport, discuss expectations and concerns, and set intentions for the treatment. During the administration stage, the patient will be administered a dose of the psychedelic substance, either orally, transdermally, intranasaly, intravenously, intramuscularly, or the like, under the supervision of the clinician (e.g., within a clinical or other indoor environment, or in an outdoor environment), to thereby induce a psychedelic experience. In some cases, there may be multiple sessions of administration. Subsequent to the psychedelic experience, during the integration stage, the patient and clinician will work together to extract insights from the psychedelic experience(s) and integrate those insights into the patient's life. Regardless of the particular psychedelic-assisted treatment, the patient will receive a psychedelic substance to induce a psychedelic experience.

The psychedelic experience is a temporary and dissociative, altered state of consciousness, which may include altered senses of time, perception, reality, mortality, and/or the like. Experiences that might be characterized as positive typically include alterations in perception that are described as an awareness of interconnectedness between all beings, a transcendental perspective of life and the meaning of life, a significant increase in the awareness of empathy, an overall feeling of being untethered to the physical body, and a subjective loss of self-identity, which was previously experienced as the root of problematic and stigmatized symptoms that the patient encountered in the normal or unaltered state. Psychedelic experiences lack predictability and can be heavily influenced by a number of factors, including those commonly referred to as “set and setting.” The set and setting include, without limitation, the patient's history (e.g., relating to unresolved and/or unrecognized trauma), the patient's mood and/or state of mind prior to the psychedelic experience and in daily life (e.g., the patient's ability to consistently orient to time and place), the patient's general state of health (e.g., psychedelic experiences are ill-advised and potentially dangerous for those with certain disease states, such as those entailing psychosis, dementia, or prior tendencies to disconnect or disassociate from reality), the patient's intentions and expectations regarding the psychedelic experience, the physical environment in which the psychedelic experience will occur, and the patient's prior experience with psychedelics (e.g., a patient who has not previously experienced a psychedelic substance will likely differentiate from a patient with significance prior experience).

Psychedelic experiences can be euphoric, pleasurable, and profoundly meaningful to patients. Sometimes they are described as “awe-inspiring” or even as a connection to the divine. This category of psychedelic experience is often referred to as a “good trip.” Psychedelic experiences can also be isolative, confusing, overwhelming, and frightening. This category of psychedelic experiences is often referred to as a “bad trip.” However, research indicates that even a bad trip or otherwise negative psychedelic experience can produce positive changes in the prefrontal cortex of the patient's brain. This is because many psychedelic substances engage physiological processes that create new brain pathways and synapses, and perform other neurogenerative functions.

Currently, the most widely available and commonly used modalities for measuring a patient's baseline and progress are framed as enumerative scales. These measurements are only offered through a series of predetermined, symptom-based questions. These questions are intended to assess the frequency and duration of maladaptive behaviors, as symptoms which are negatively associated with mental health diagnoses and which hold the same negative stigma that mental health diagnoses often hold. Patients are asked to reflect upon and self-assess their behaviors, such as changes in sleep habits, eating habits, substance abuse, negative thoughts, motivation levels, energy levels, frequency of thoughts, impulses, or occurrences of self-harm, and the like. All of these behaviors relate to negativity and stigmatization, from which the patient is hoping to be disassociated. By virtue of this self-reporting, patients may infer that they are unhealed, sick, or “broken.” Ultimately, the very measures upon which a patient is asked to reflect become triggers for negativity, regression, and potential self-harm, since the measures incorporate the very behaviors and symptoms that the patient is attempting to leave behind in a quest for change and healing, and which are a reminder of the options of self-harm and suicide. In addition, current self-reporting measures do not invite the patient to recognize, reflect upon, or utilize the unique and profound mechanism of the psychedelic experience to release the old self concept and sense of ego, which was not serving the patient and may have been causing problematic behaviors. The old self, with which a patient identifies prior to a psychedelic experience, is commonly challenged by behaviors and affective states that result in mental health diagnoses.

The inventor has recognized that, during psychedelic-assisted treatment, after a patient transitions from the dissociative, altered state of the psychedelic experience back to a normal (i.e., unaltered or baseline) state, it is beneficial for the clinician to, not only comfort the patient and ensure the patient's safety, but also guide the patient towards a positive state of mind and begin the integration process by extracting meaning (e.g., self-reflection, a new perspective of the self, etc.) from the psychedelic experience. The inventor has also recognized that such an interaction is effective for other types of therapeutic treatments, besides psychedelic-assisted treatment, such as in the case of therapy for a minor.

### SUMMARY

Accordingly, processes and implements are disclosed for psychedelic-assisted or other therapeutic treatment that produces improved outcomes.

In an embodiment, a psychedelic-assisted treatment comprises: administering an effective dose of a psychedelic substance to a patient to induce a psychedelic experience by the patient, wherein the psychedelic experience comprises an altered state of the patient; monitoring the patient during the psychedelic experience; and after egress by the patient from the altered state to a normal state, engaging the patient with a visual implement that comprises visual content, wherein engaging the patient comprises prompting the patient to identify, outline, and describe a feature of the visual content.

The visual content may comprise at least one object within an environment and a plurality of colors. The at least one object may be an animal. Engaging the patient with the visual implement may comprise: prompting the patient to identify the feature of the visual content; prompting the patient to relate the feature of the visual content; prompting the patient to outline the feature of the visual content; and prompting the patient to narrate the feature of the visual content.

The visual content may be textured. The visual content may be a painting. The feature may be a visual or tactile feature of the textured visual content. Engaging the patient with the visual implement may comprise: prompting the patient to find the feature of the visual content; prompting the patient to outline the feature of the visual content; prompting the patient to recognize the feature of the visual content; prompting the patient to reflect on the feature of the visual content; and guiding the patient to release anxiety and elevate perspective by telling a positive story using the feature of the visual content.

Engaging the patient may further comprise: instructing the patient to draw a drawing on the visual implement to produce the visual content; prompting the patient to identify a picture, as the feature, in the drawing; prompting the patient to outline the picture; and prompting the patient to describe the picture. Prompting the patient to identify a picture may comprise instructing the patient to rotate the drawing to identify the picture. Instructing the patient to draw a drawing may comprise instructing the patient to sign the patient's name. Instructing the patient to draw a drawing may comprise instructing the patient to scribble on the visual implement.

The visual implement may be a non-electronic substrate, and the visual content may be on a surface of the non-electronic substrate that is configured to be drawn upon.

The visual implement may be a processing device with an integrated touch-panel display that displays the visual content, receives the outline as an input, and stores the outline in association with the visual content in a memory of the processing device. The processing device may be a tablet computer that comprises: at least one hardware processor; a non-transitory computer-readable medium; and a software application, stored on the non-transitory computer-readable medium, wherein the software application is configured to, when executed by the at least one hardware processor, receive an input from a user, in response to the input, display the visual content in a graphical user interface of the software application, and, after displaying the visual content, receive the outline as the input via the graphical user interface, and store the outline in association with the visual content in the non-transitory computer-readable medium as the memory. The outline may be received as a sequence of strokes, and the psychedelic-assisted treatment may further comprise, by the software application: mapping each stroke in the sequence of strokes to a coordinate system of the visual content and a temporal position with the sequence of strokes, and in response to a user operation, playing back the sequence of strokes. The psychedelic-assisted treatment may further comprise, by the software application: displaying a list of available visual content in the graphical user interface; and as the input, receiving a selection of the visual content from the list of available visual content. Prompting the patient may comprise one or both of displaying or playing audio of one or more prompts via the graphical user interface.

The psychedelic-assisted treatment may further comprise, before administering the effective dose of the psychedelic substance to the patient, engaging the patient with the visual implement to establish a baseline status of the patient. The psychedelic-assisted treatment may further comprise, before engaging the patient with the visual implement to establish the baseline status of the patient, triggering the patient with at least one stimulus.

In an embodiment, a therapeutic treatment comprises engaging a patient with a visual implement that comprises visual content, wherein engaging the patient comprises prompting the patient to identify, outline, and describe a feature of the visual content.

It should be understood that any of the features in the methods above may be implemented individually or with any subset of the other features in any combination. Thus, to the extent that the appended claims would suggest particular dependencies between features, disclosed embodiments are not limited to these particular dependencies. Rather, any of the features described herein may be combined with any other feature described herein, or implemented without any one or more other features described herein, in any combination of features whatsoever.

### BRIEF DESCRIPTION OF THE DRAWINGS

The details of the present invention may be gleaned in part by study of the accompanying drawings, in which like reference numerals refer to like parts, and in which:

FIG. 1 illustrates an example process for a therapeutic (e.g., psychedelic-assisted), according to an embodiment;

FIG. 2 illustrates an example processing system that may be used as a visual implement during a therapeutic (e.g., psychedelic-assisted) treatment, according to an embodiment;

FIG. 3 illustrates a first example of a visual implement that may be used during a therapeutic (e.g., psychedelic-assisted) treatment, according to an embodiment;

FIG. 4 illustrates an example process for using the first example of the visual implement during a therapeutic (e.g., psychedelic-assisted) treatment, according to an embodiment;

FIG. 5 illustrates a second example of a visual implement that may be used during a therapeutic (e.g., psychedelic-assisted) treatment, according to an embodiment;

FIG. 6 illustrates an example process for using the second example of the visual implement during a therapeutic (e.g., psychedelic-assisted) treatment, according to an embodiment;

FIGS. 7A-7D illustrate a third example of a visual implement that may be used during a therapeutic (e.g., psychedelic-assisted) treatment, according to an embodiment; and

FIG. 8 illustrates an example process for using the third example of the visual implement during a therapeutic (e.g., psychedelic-assisted) treatment, according to an embodiment.

### DETAILED DESCRIPTION

Embodiments of processes and implements for an improved method of a therapeutic (e.g., psychedelic-assisted) treatment are disclosed. After reading this description, it will become apparent to one skilled in the art how to implement the invention in various alternative embodiments and alternative applications. However, although various embodiments of the present invention will be described herein, it is understood that these embodiments are presented by way of example and illustration only, and not limitation. As such, this detailed description of various embodiments should not be construed to limit the scope or breadth of the present invention as set forth in the appended claims.

### 1. Psychedelic-Assisted Treatment

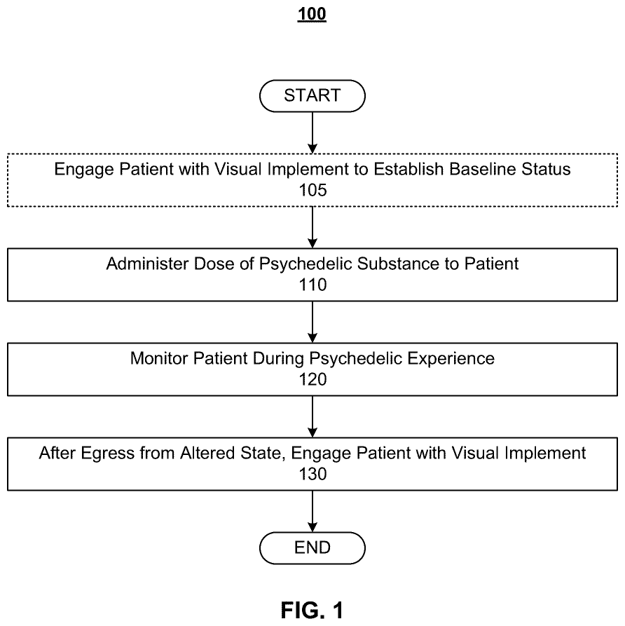

FIG. 1 illustrates an example process 100 for a therapeutic treatment, according to an embodiment. In the illustrated embodiment, it is assumed that the therapeutic treatment is a psychedelic-assisted treatment. However, it should be understood that the therapeutic treatment could be administered utilizing a non-psychedelic substance. In this case, the psychedelic substance, described with respect to any of the subprocesses of process 100, may be replaced with a non-psychedelic substance. Alternatively, the therapeutic treatment could be administered with no substance at all. In this case, any subprocess of process 100 which utilizes a substance may be omitted from process 100.

Process 100 may be performed by a clinician or other attendant to the patient. For simplicity, the attendant will simply be referred to herein as a “clinician,” regardless of their particular profession, expertise, or relationship to the patient. The clinician may be a therapist, and process 100 may be performed as part of a psychedelic-assisted therapy. Alternatively, the clinician may be another healthcare professional (e.g., doctor, nurse, etc.) and/or process 100 may be performed as part of another type of psychedelic-assisted treatment. Ideally, process 100 is performed within a clinical environment, such as a hospital, medical clinic, or other healthcare facility. However, this is not a requirement of any embodiment. In alternative embodiments, the clinician may be someone other than a healthcare professional (e.g., a friend, family member, spiritual adviser, etc.) and/or the environment may be somewhere other than a clinical environment (e.g., at a home, in the outdoors, etc.). Also, it should be understood that the clinician may not necessarily be the same person throughout the entire process 100. For example, a first clinician (e.g., psychiatric nurse) may administer the psychedelic substance, a second clinician (e.g., psychiatric nurse) may monitor the patient, and/or a third clinician (e.g., psychiatrist) may engage with the patient after the psychedelic experience.

In an initial subprocess 105, the clinician may engage the patient with a visual implement, as is described in greater detail elsewhere herein, to establish a baseline status for the patient. A baseline status may be useful as a comparison against the patient's status during and after the psychedelic experience. A trigger may be used in subprocess 105, as is discussed in greater detail elsewhere herein, to stimulate the patient prior to establishing the baseline status. In an alternative embodiment, the use of a visual implement in subprocess 105 may be omitted, the use of a trigger may be omitted, or subprocess 105 may be omitted altogether.

In preparation for the psychedelic experience, the patient may be placed in a comfortable, private, indoor space. The patient may be seated in a comfortable chair, placed in a supine position on a bed, or the like. The patient's eyes may be covered by an eye mask configured to block out light. The patient may also be fitted with a pair of headphones configured to block out ambient noise and/or output appropriate music or other suitable sounds (e.g., selected by the patient and/or clinician) during the psychedelic experience. It should be understood that this is simply one example of the environment in a clinical setting, and that the other environments are possible, including in non-clinical settings.

In subprocess 110, an effective dose of a psychedelic substance is administered to the patient. The psychedelic substance may be administered orally (e.g., swallowed, smoked, chewed, etc.), transdermally, intranasaly, intravenously (e.g., via injection), intramuscularly, or in any other suitable manner. It should be understood that the effective dose may depend on the psychedelic substance, the patient (e.g., medical condition, body mass, tolerance, age, etc.), the method of administration, the goals of the treatment, and/or the like. As examples, typical effective doses include, without limitation: 50-200 micrograms (μg) per individual for LSD; 20-30 milligrams (mg) per 70 kilograms (kg) of body mass for psilocybin; 0.5-1.0 mg per 1 kg of body mass for ayahuasca; 200-400 mg per individual (if administered orally) for mescaline; 20-40 mg per individual (if smoked) or 0.2-0.4 mg per 1 kg of body mass (if administered intravenously) for DMT; 25-100 mg per individual (if administered intravenously), 75-125 mg per individual (if administered intramuscularly), or 200-300 mg per individual (if administered orally) for ketamine; 500-1,000 mg per individual (if administered orally) for ibogaine; 3 mg per 1 kg of body mass for MDMA; 0.2-1.0 mg per individual (if smoked) for salvinorin A; and 66-800 mg per individual (if smoked) or 5-25 mg per individual (if ingested) for THC.

In subprocess 120, the patient may be monitored during the psychedelic experience. For example, the clinician may remain physically present with the patient, maintain visual contact with the patient, provide continual check-ins, and/or the like, throughout the patient's psychedelic experience, to ensure the patient's safety and/or comfort. The clinician may also provide emotional support, physical assistance, and/or the like to the patient, if necessary. It should be understood that the length of the psychedelic experience will depend on the dose that was administered, the method of administration, the psychedelic substance, the patient, and/or the like.

During the psychedelic experience, the patient will enter an altered state, and remain in this altered state until the effects of the psychedelic substance begin to wear off, as the psychedelic substance metabolizes. As mentioned above, the psychedelic experience may represent a good trip or a bad trip. As the effects of the psychedelic substance wear off, the patient will egress from the altered state and transition back to a normal state.

In subprocess 130, after the patient egresses from the altered state, the clinician may engage the patient with a visual implement. In an embodiment that includes subprocess 105, the visual implement may be the same visual implement that was used in subprocess 105 to establish the baseline status of the patient. The visual implement represents a “language” that the patient can use to effectively express or exude aspects of the patient's psychedelic experience and/or the patient's current perspective, following the psychedelic experience, to the clinician. In an embodiment, the visual implement, along with the strategy employed by the clinician, is designed to provide comfort to the patient, as well as assistance with communication and orientation to the present moment if the patient is experiencing difficulty readjusting and/or acclimating. In essence, the visual implement and accompanying strategy may provide a container for the patient to positively express the psychedelic experience, regardless of whether the psychedelic experience was good or bad. As a result, the patient is provided with an opportunity to engage with the present moment and environment, as well as communicate with the clinician in a manner that offers safety, comfort, creative self-expression and reflection, to thereby improve the psychedelic-assisted treatment and its outcome.

Subprocess 130 is described as occurring after the patient has egressed from the altered state. The patient's return to the normal, unaltered state may be determined by comparing the patient's vital signs to a set of baseline vital signs established before the psychedelic experience. Examples of vital signs include, without limitation, blood pressure, heart rate, oxygen saturation, and/or the like. Thus, when the patient's post-egress vital signs match those within the patient's baseline vital signs (e.g., within a tolerance), the clinician may determine that the patient has returned to the normal, unaltered state, and initiate subprocess 130.

In an alternative embodiment, subprocess 130 could be performed as the patient is transitioning from the altered state of the psychedelic experience to the unaltered state. Under current practice, it is generally not advisable to interfere with a patient until after the transition to the unaltered state is complete. This is because interference may affect and possibly interrupt or damage the therapeutic aspect of the patient's “physiologic arrival” and realignment with body, self, and identity. However, if the patient is physiologically stable (i.e., the patient's vital signs are stable), but is struggling with the process of reorientation, such that a private, quiet, therapeutic “comedown” is proving to be problematic, the clinician may initiate subprocess 130 at that time, which may be prior to complete egress. Additionally, as the understanding of psychedelic-assisted treatment evolves, if it turns out that engagement with the patient during the transition from the altered state to the unaltered state has some benefits, subprocess 130 may be initiated at some point during the transition from the altered state to the unaltered state, instead of waiting for the patient to be in the unaltered state. Similarly, in a non-clinical setting, such as an outdoor or recreational environment (e.g., in which vital signs cannot be assessed), subprocess 130 may be initiated at some point during the transition from the altered state to the unaltered state.

In an embodiment in which no substance is utilized, process 100 may omit subprocesses 105, 110, and 120. In this case, process 100 may consist of only subprocess 130, or may comprise subprocess 130 in combination one or more other subprocesses preceding and/or following subprocess 130. In other words, the clinician may engage the patient with the visual implement, without the patient having been administered any substance. Such an embodiment may be particularly suited for use with a patient who is a minor (i.e., a toddler, child, teenager, or other adolescent under the age of eighteen), for which a psychedelic or other substance is not appropriate, recommended, or well-studied.

### 2. Example Processing System

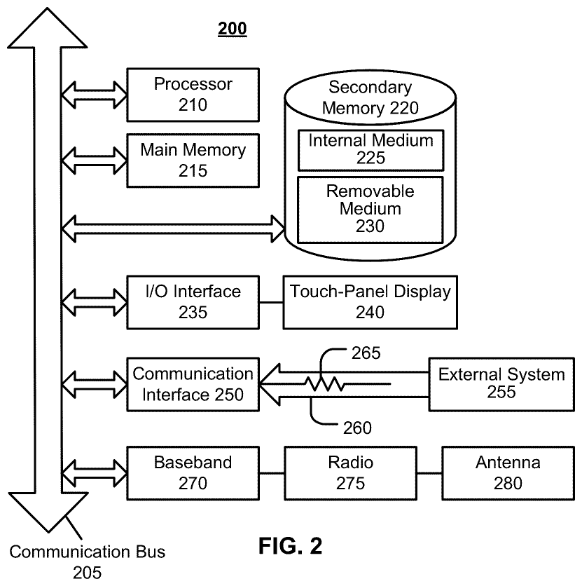

FIG. 2 is a block diagram illustrating an example of a wired or wireless system 200 that may be used as the visual implement in one or more embodiments. System 200 can be a server, conventional personal computer, or any other processor-enabled device that is capable of wired or wireless data communication. However, it is generally contemplated that system 200 would be a handheld tablet computer (e.g., smartphone or larger device), with which a patient can easily interact, even while the patient is in a dissociative state.

System 200 comprises one or more processors 210. Processor(s) 210 may comprise a central processing unit (CPU). Additional processors may be provided, such as a graphics processing unit (GPU), an auxiliary processor to manage input/output, an auxiliary processor to perform floating-point mathematical operations, a special-purpose microprocessor having an architecture suitable for fast execution of signal-processing algorithms (e.g., digital-signal processor), a subordinate processor that is subordinate to the main processing system (e.g., back-end processor), an additional microprocessor or controller for dual or multiple processor systems, and/or a coprocessor. Such auxiliary processors may be discrete processors or may be integrated with a main processor 210. Examples of processors which may be used in system 200 include, without limitation, any of the processors (e.g., Pentium™, Core i7™, Xeon™, etc.) available from Intel Corporation of Santa Clara, California, any of the processors available from Advanced Micro Devices, Incorporated (AMD) of Santa Clara, California, any of the processors (e.g., A series, M series, etc.) available from Apple Inc. of Cupertino, any of the processors (e.g., Exynos™) available from Samsung Electronics Co., Ltd., of Seoul, South Korea, any of the processors available from NXP Semiconductors N.V. of Eindhoven, Netherlands, and/or the like.

Processor 210 is preferably connected to a communication bus 205. Communication bus 205 may include a data channel for facilitating information transfer between storage and other peripheral components of system 200. Furthermore, communication bus 205 may provide a set of signals used for communication with processor 210, including a data bus, address bus, and/or control bus (not shown). Communication bus 205 may comprise any standard or non-standard bus architecture such as, for example, bus architectures compliant with industry standard architecture (ISA), extended industry standard architecture (EISA), Micro Channel Architecture (MCA), peripheral component interconnect (PCI) local bus, standards promulgated by the Institute of Electrical and Electronics Engineers (IEEE) including IEEE 488 general-purpose interface bus (GPIB), IEEE 696/S-100, and/or the like.

System 200 may comprise a main memory 215. Main memory 215 provides storage of instructions and data for software executing on processor 210. It should be understood that software stored in the memory and executed by processor 210 may be written and/or compiled according to any suitable language, including without limitation C/C++, Java, JavaScript, Perl, Visual Basic, .NET, and the like. Main memory 215 is typically semiconductor-based memory such as dynamic random access memory (DRAM) and/or static random access memory (SRAM). Other semiconductor-based memory types include, for example, synchronous dynamic random access memory (SDRAM), Rambus dynamic random access memory (RDRAM), ferroelectric random access memory (FRAM), and the like, including read only memory (ROM).

System 200 may comprise a secondary memory 220. Secondary memory 220 is a non-transitory computer-readable medium having computer-executable code and/or other data stored thereon. This software, stored on secondary memory 220, is read into main memory 215 for execution by processor 210. Secondary memory 220 may include, for example, semiconductor-based memory, such as programmable read-only memory (PROM), erasable programmable read-only memory (EPROM), electrically erasable read-only memory (EEPROM), and flash memory (block-oriented memory similar to EEPROM). As used herein, the term “computer-readable medium” is used to refer to any non-transitory computer-readable storage media used to provide computer-executable code and/or data to or within system 200.

Secondary memory 220 may optionally include an internal medium 225 and/or a removable medium 230. Removable medium 230 may be read from and/or written to in any well-known manner. Removable storage medium 230 may be, for example, a magnetic tape drive, a compact disc (CD) drive, a digital versatile disc (DVD) drive, other optical drive, a flash memory drive, and/or the like.

System 200 may comprise an input/output (I/O) interface 235. I/O interface 235 provides an interface between one or more components of system 200 and one or more input and/or output devices. Example input devices include, without limitation, sensors, keyboards, touch screens or other touch-sensitive devices, cameras, biometric sensing devices, computer mice, trackballs, pen-based pointing devices, and/or the like. Examples of output devices include, without limitation, other processing devices, cathode ray tubes (CRTs), plasma displays, light-emitting diode (LED) displays, liquid crystal displays (LCDs), printers, vacuum fluorescent displays (VFDs), surface-conduction electron-emitter displays (SEDs), field emission displays (FEDs), and/or the like.

In some cases, an input and output device may be combined, such as in the case of a touch-panel display 240. Advantageously, the incorporation of a touch-panel display 240 into system 200, as the visual implement, will allow a patient to interact with the visual implement in an easy and intuitive manner. This can be especially important when the patient is in a dissociative state, transitioning out of a dissociative state, or has just egressed from a dissociative state. Thus, in an embodiment in which the visual implement is a system 200, the visual implement is preferably a tablet computer (e.g., smartphone or larger device) with an integrated touch-panel display 240.

System 200 may include a communication interface 250. Communication interface 250 allows software (e.g., computer-executable code and/or data) to be transferred between system 200 and external devices (e.g. printers), networks, or other information sources. For example, software may be transferred to system 200 from an external system 255. External system 255 may comprise an external storage medium, another system 200, a network server, and/or the like. Examples of communication interface 250 include a built-in network adapter, network interface card (NIC), Personal Computer Memory Card International Association (PCMCIA) network card, card bus network adapter, wireless network adapter, Universal Serial Bus (USB) network adapter, modem, a wireless data card, a communications port, an infrared interface, an IEEE 1394 fire-wire, and any other device capable of interfacing system 200 with a network or other external system. Communication interface 250 preferably implements industry-promulgated protocol standards, such as Ethernet IEEE 802 standards, Fiber Channel, digital subscriber line (DSL), asynchronous digital subscriber line (ADSL), frame relay, asynchronous transfer mode (ATM), integrated digital services network (ISDN), personal communications services (PCS), transmission control protocol/Internet protocol (TCP/IP), serial line Internet protocol/point to point protocol (SLIP/PPP), and so on, but may also implement customized or non-standard interface protocols as well.

Software transferred via communication interface 250 is generally in the form of electrical communication signals 265. These signals 265 may be provided to communication interface 250 via a communication channel 260. Communication channel 260 may be a wired or wireless network, or any variety of other communication links. Communication channel 260 carries signals 265 and can be implemented using a variety of wired or wireless communication means including wire or cable, fiber optics, conventional phone line, cellular phone link, wireless data communication link, radio frequency (“RF”) link, or infrared link, just to name a few.

System 200 may also include wireless communication components that facilitate wireless communication over a voice network and/or a data network. The wireless communication components comprise an antenna system 280, a radio system 275, and a baseband system 270. In system 200, radio frequency (RF) signals are transmitted and received over the air by antenna system 280 under the management of radio system 275.

In an embodiment, antenna system 280 may comprise one or more antennae and one or more multiplexors (not shown) that perform a switching function to provide antenna system 280 with transmit and receive signal paths. In the receive path, received RF signals can be coupled from a multiplexor to a low noise amplifier (not shown) that amplifies the received RF signal and sends the amplified signal to radio system 275.

In an alternative embodiment, radio system 275 may comprise one or more radios that are configured to communicate over various frequencies. In an embodiment, radio system 275 may combine a demodulator (not shown) and modulator (not shown) in one integrated circuit (IC). The demodulator and modulator can also be separate components. In the incoming path, the demodulator strips away the RF carrier signal leaving a baseband receive signal, which is sent from radio system 275 to baseband system 270.

### 3. Visual Implement

Embodiments of the visual implement that may be used in subprocess 130 of psychedelic-assisted treatment 100 will now be described with reference to specific examples. It should be understood that the illustrated examples are not limiting. Rather, the examples are used to demonstrate the general attributes of a suitable visual implement in various embodiments. The present disclosure contemplates any visual implement that possesses one or more of the general attributes of these specific examples.

In an embodiment, each visual implement may comprise a substrate on which the visual content in the described examples is printed, drawn, painted, etched, glued, projected, and/or the like. It should be understood that the substrate may be any type of substrate that is suitable for bearing the visual content. Examples of substrates include, without limitation, paper, a canvas, a poster board, a white board, cardboard, a wall, or the like. In an embodiment, the visual implement is able to be held by a single human hand or pair of human hands. Additionally or alternatively, the visual implement is capable of being drawn upon. The visual implement may be disposable (e.g., for use by a single patient and/or in a single session) or non-disposable and reusable (e.g., for use by a plurality of patients and/or in a plurality of sessions).

In this embodiment, in any of the described examples, when a patient draws on a visual implement (e.g., to outline a feature in the visual content), the patient may do so with a writing instrument. Examples of writing instruments include, without limitation, a pencil, a pen, a paintbrush, a highlighter (e.g., yellow highlighter), a permanent marker, a dry-erase marker, or the like. While the patient could also draw with a non-writing instrument, such as the patient's finger or a stylus, the resulting drawing would not generally be persistently visible for future reference. Therefore, a writing instrument is preferred.

In an alternative embodiment, each visual implement may comprise a system 200, such as a tablet computer, that displays the visual content in any of the described examples. In this case, disclosed embodiments may be implemented as a software application that executes locally on processor(s) 210 of system 200, executes remotely on an external system 255, or is distributed across system 200 and external system 255, such that some functions are executed locally on system 200 while other functions are executed remotely on external system 255. In any case, the software application may generate a graphical user interface that includes one or more screens. These screens may comprise a combination of content and elements, such as text, images, videos, animations, references (e.g., hyperlinks), frames, inputs (e.g., textboxes, text areas, checkboxes, radio buttons, drop-down menus, buttons, forms, etc.), scripts (e.g., JavaScript), and the like, including elements comprising or derived from data stored in local memory (e.g., main memory 215 and/or secondary memory 220) or remote memory (e.g., of external system 255). These screens may comprise menus or other navigational elements that enable a user (e.g., clinician or patient) to access the various functions of disclosed embodiments, including any of the visual content described herein. In some embodiments, two or more screens may be served in the form of a wizard, in which case these screens may be served in a sequential manner, and one or more of the sequential screens may depend on an interaction of the user with one or more preceding screens.

The visual content may be stored as an image or other data in main memory 215, secondary memory 220, and/or remote memory. During use, the software application may retrieve the visual content from the memory and display the visual content on touch-panel display 240, to turn system 200 into the visual implement. In embodiments which prompt the user for inputs, the prompts may be displayed as text on touch-panel display 240 and/or played from a speaker of system 200 (e.g., as synthesized or recorded audio), inputs may be received via touch-panel display 240 (e.g., via contact with a touch sensor in touch-panel display 240), and the inputs may be processed by processor(s) 210 and/or represented in a data structure that is stored in main memory 215 and/or secondary memory 220.

In an embodiment, the particular visual content, to be used in subprocess 130, may be selected from a plurality of available visual content. For example, the user (e.g., clinician or patient) may select the visual content to be used before or during subprocess 105 or at the preparation stage of a psychedelic-assisted therapy session. In this case, the most appropriate visual content may be selected based on the patient's desire, the clinician's experience, the goals of the treatment, and/or the like. Thus, it should be understood that different visual content may be selected for different patients, as well as for different sessions with the same patient. In an embodiment, the visual content may be selected by the clinician without being exposed to the patient until subprocess 105 or subprocess 130, such that the patient will not see the visual content before subprocess 105 or subprocess 130.

In an embodiment, the software application implements all of the various examples of the visual implement described herein. For example, all of the examples of the visual content and associated functions, described herein, may be provided within a single software application. The user (e.g., clinician or patient) may select the particular visual content to be used, and the software application will execute the functions necessary to convert system 200 into the visual implement that is suitable for that selected visual content, including any necessary screens, wizards, drawing functions, writing functions, and/or the like.

In any case, the selected visual content is retrieved and utilized in subprocess 130, along with any associated functions, and any of the disclosed strategies. In the event that the visual implement is a non-electronic substrate, a visual implement may be constructed for each of the plurality of available visual content, and the visual implement with the selected visual content may be used in subprocess 105 and retained for subprocess 130. In the event that the visual implement comprises a system 200, the visual content may retrieved from memory (e.g., secondary memory 220) and displayed on touch-panel display 240 during subprocess 105 and subprocess 130.

In any embodiment in which the visual implement comprises a system 200, in any of the described examples, when a patient draws on the visual implement (e.g., to outline a feature in the visual content), the patient may do so with the patient's finger or a stylus. The drawing may be displayed, in real time, as the patient draws it, as an overlay over the existing visual content. In addition, system 200 may receive the drawing and store it in non-persistent memory (e.g., main memory 215) and/or persistent memory (e.g., secondary memory 220), so that the drawing can be replicated in the future. In an embodiment, the relationship of the drawing to the existing visual content may be maintained (e.g., via identical coordinate systems or some other form of mapping, or as a composite image), such that the drawing, in the context of the existing visual content, can be replicated in the future. In a further embodiment, the strokes of any drawing may be mapped to the visual content in sequence, such that the drawing may be played back stroke by stroke. In other words, each stroke (e.g., from the start of a contact to the end of that contact) may be mapped to the coordinate system of the visual content, as well as to a temporal position within the sequence of strokes.

In an embodiment, the colors of the visual content and the patient's drawing may be complementary. For example, the visual content may be generally or primarily blue, whereas the patient's drawing may be transparent yellow, such as would be produced by a yellow highlighter. The use of transparent highlighting over a complementary color produces a new color that is easily discernable and trackable. For instance, in the case of a blue background and yellow highlighting, the patient's drawing will be green (i.e., a mixture of blue and yellow). It should be understood that such an embodiment may pertain both to a non-electronic visual implement (e.g., in which case an actual yellow highlighter may be used) and an electronic visual implement comprising system 200 (e.g., in which case, the patient's drawing may be overlaid as transparent yellow highlighting).

Although each of the examples of the visual implement will be described herein with respect to a specific strategy, implemented as a process, any one of the visual implements may be used with any one of the strategies. Thus, it should be understood that a clinician may mix and match any particular visual implement with any particular strategy, to achieve the desired objectives for the psychedelic-assisted treatment. These selections may be performed prior to subprocess 130 (e.g., in subprocess 105) or may be performed on the fly, for example, based on the clinician's observations of the patient's state in subprocess 130 (e.g., whether the patient has had a good trip or a bad trip).

### 3.1. First Example

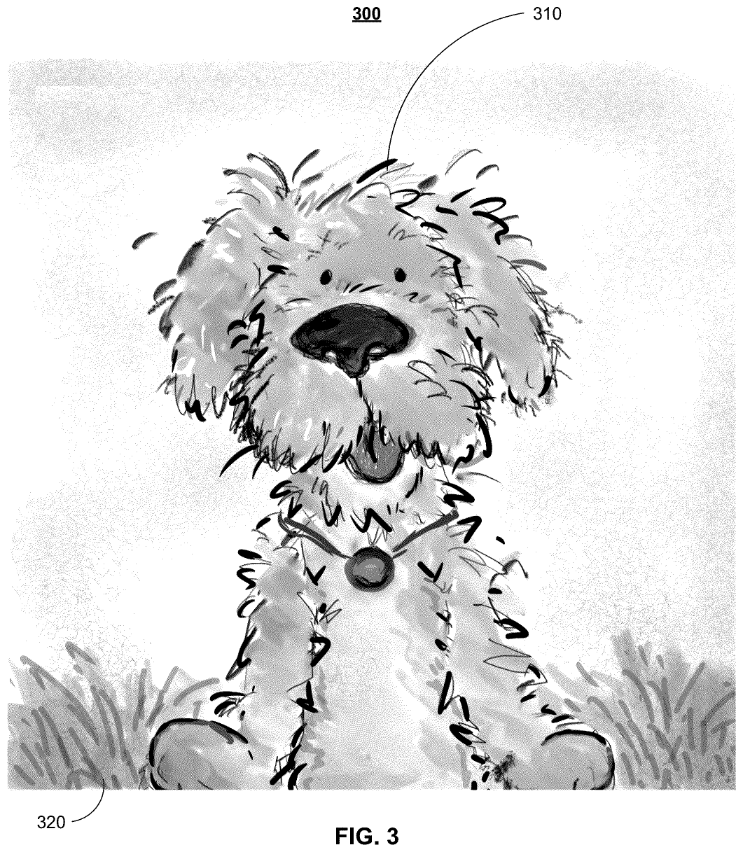

FIG. 3 illustrates a first example of a visual implement 300 that may be used in subprocess 130, according to an embodiment. At a high level, the visual content of visual implement 300 comprises a caricature of a comforting object 310, within a comforting environment 320. The surface of visual implement 300, on which the visual content is present (e.g., drawn, painted, displayed, etc.), may be smooth and untextured. Alternatively, the surface of visual implement 300, on which the visual content is present, could be textured.

In the illustrated example, object 310 comprises an animal, and particularly, a friendly dog. Environment 320 comprises grass and a sunny sky. It should be understood that these are simply examples, and that object 310 may comprise other animals, people, inanimate things, and/or the like. Similarly, environment 320 may comprise other environments, including indoor environments, outdoor environments, surreal environments, and/or the like, or may simply comprise a background color or colors. While only a single object 310 is illustrated in a single environment 320, visual implement 300 could comprise a plurality of objects 310 and/or a plurality of environments 320.

Object 310 and/or environment 320 may be depicted in any style, including cartoonish, realistic, or the like. In general, object 310 and environment 320 should be comforting and non-threatening to convey that the patient is in a safe environment. For example, object 310 may be a cute, cartoonish, or otherwise friendly looking dog, cat, or other animal. In addition, object 310 may be colored in a soft, comforting color, such as yellow. Similarly, environment 320 may be colored with soft hues, such as light blues and/or greens.

During subprocess 130, visual implement 300 may be shown to the patient. For example, the patient and/or clinician may hold visual implement 300, such that the visual content of visual implement 300 is visible to the patient. As the patient views the visual content, the clinician may gently guide the patient through a description of what the patient sees. In particular, the patient may look at object 310 and/or environment 320, to evaluate the shapes and colors and convey what the patient sees to the clinician. This exercise can remind the patient that the patient is in the patient's body, as they transition out of the dissociative state of the psychedelic experience. The goal of the clinician in subprocess 130 is as a guide to help the patient describe and evaluate the psychedelic experience via visual implement 300.

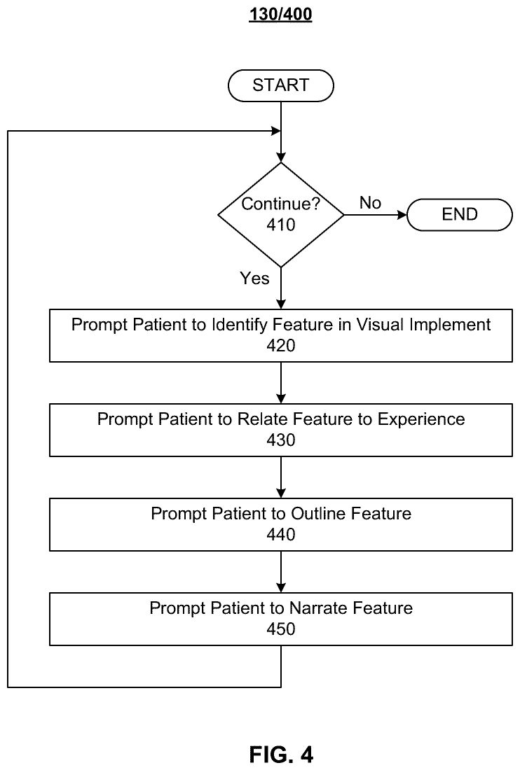

FIG. 4 illustrates an example of subprocess 130, utilizing visual implement 300, according to an embodiment. This embodiment of subprocess 130 may also be referred to herein as process 400, when differentiation from other embodiments is desired. In this embodiment, the clinician may engage the patient using an Identify-Relate-Outline-Narrate (IRON) strategy, implemented as process 400. The goal of the IRON strategy is to focus the patient on positive aspects of the psychedelic experience to guide the patient towards a positive state of mind, so that the patient feels safe, comfortable, and grounded as the patient returns to the present moment. This is especially important if the clinician senses that the patient has experienced a bad trip. The clinician may sense that the patient has experienced a bad trip if the patient is negative or anxious during the engagement in subprocess 130. The IRON strategy will give the clinician a good sense of the patient's mental state after the psychedelic experience, which will allow the clinician to determine whether or not it is safe to release the patient (e.g., end the session, allow the patient to exit the treatment facility, etc.). It should be understood that, in an alternative embodiment in which process 100 does not utilize a psychedelic substance, the IRON strategy can still be used to gauge the patient's mental state, even though there has been no psychedelic experience. In other words, the IRON strategy can be used regardless of whether or not there has been a psychedelic experience or the patient has been in a dissociative state.

The IRON strategy in process 400 is illustrated as iterative and structured. However, the IRON strategy may, instead, be more free-flowing and unstructured, with some of the stages being performed in different orders than illustrated and/or in parallel, and/or one or more of the stages being skipped or omitted. In either case, the clinician or software of system 200 may continue process 400 for as long it is deemed beneficial (i.e., “Yes” in decision stage 410), and end process 400 once it is deemed to no longer be beneficial (i.e., “No” in decision stage 410). Process 400 may be deemed to no longer be beneficial once no new insights are obtainable.

In the identify stage 420, the clinician or the software of system 200 may prompt the patient to identify some feature in the visual content of visual implement 300. The patient may identify first object 310, environment 320, and/or second object 330, or some aspect of one of these things. For example, in the example in which first object 310 is a dog, the patient may identify the dog or may identify some part of the dog, such as the dog's fur, eyes, nose, ears, face, feet, and/or the like.

In the relate stage 430, the clinician or the software of system 200 may prompt the patient to relate a feature, identified in stage 420, to the patient's psychedelic experience. In other words, stage 430 influences the patient to identify a relationship between the visual content of visual implement 300 and the patient's psychedelic experience and/or orient the patient towards the present moment to determine the patient's perspective.

In the outline stage 440, the clinician or the software of system 200 may prompt the patient to outline the feature, identified in stage 420. In the event that visual implement 300 is non-electronic, the patient may use a writing instrument (e.g., pen, pencil, highlighter, marker, etc.) to outline the feature. In the event that visual implement 300 comprises system 200, the patient may use the patient's finger or a stylus to outline the feature in the visual content displayed on touch-panel display 240. In this case, the outline may be overlaid, in real time, as the patient draws it, on the visual content displayed on touch-panel display 240. In addition, the outline may be stored in memory (e.g., main memory 215 and/or secondary memory 220) for future reference.

In the narrate stage 450, the clinician or the software of system 200 may prompt the patient to narrate the feature, identified in stage 420. This narration by the patient may reflect what the patient is currently experiencing (e.g., “where” the patient is) or previously experienced during the psychedelic experience (e.g., “where” the patient was). Thus, the IRON strategy provides the clinician with a roadmap for helping the patient evaluate visual implement 300 and determine whether or not the patient is in touch with reality, while also gaining insights from the patient's psychedelic experience. In the event that visual implement 300 comprises system 200, the patient's narration may be automatically recorded, via a microphone of system 200, to patient information within memory of system 200.

### 3.2. Second Example

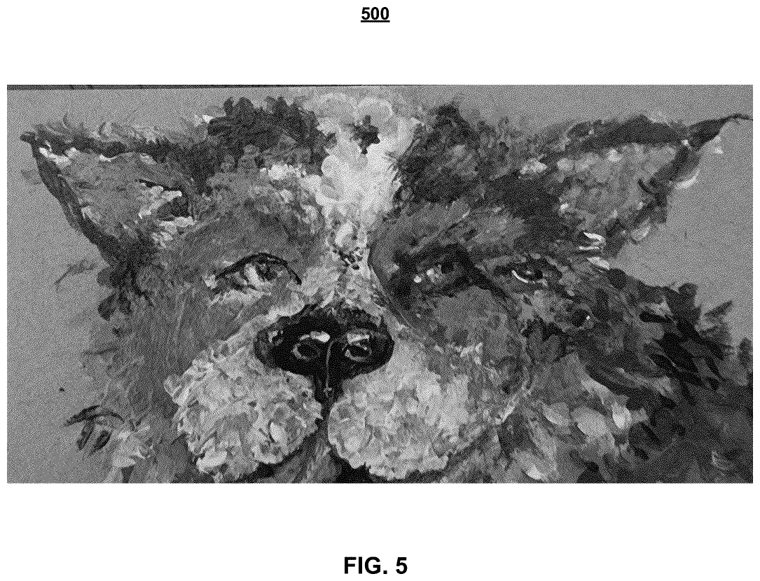

FIG. 5 illustrates a second example of a visual implement 500 that may be used in subprocess 130, according to an embodiment. At a high level, the visual content of visual implement 500 comprises a textured image. In other words, the visual content is both visual and tactile. For example, visual implement 500 may comprise a textured painting on a canvas. The painting may be of anything or nothing in particular. For example, the painting may be of one or more objects (e.g., an animal, such as a dog) or may be abstract. It should be understood that visual implement 500 may be constructed in any manner that generates texture or other tactile features. While this second example of visual implement 500 is generally contemplated to comprise a non-electronic substrate, visual implement 500 could comprise a system 200 that is constructed with tactile features on the touch-panel display 240 or elsewhere on a housing of system 200.

In an embodiment, the visual content of visual implement 500 is not exposed to the patient prior to subprocess 130. In this case, the clinician may select the visual content without the patient's direct involvement. In other words, the first time the patient sees the visual content of visual implement 500 will be during subprocess 130. Thus, the patient will not be influenced by any preconceived notions about the visual content. However, it should be understood that, in an alternative embodiment, the patient could be exposed to the visual content of visual implement 500 prior to subprocess 130 (e.g., in subprocess 105).

In the same or similar manner as with the first example, the visual content of visual implement 500 may be shown to the patient in subprocess 130, after the patient's transition from the altered state to the normal state. Because visual implement 500 is textured, the patient may engage with both visual and tactile features of visual implement 500. As the patient views and/or touches visual implement 500, the clinician may gently guide the patient through a description of what the patient sees and/or feels. Again, the goal of the clinician in subprocess 130 is as a guide to help the patient describe and evaluate the psychedelic experience, as well as integrate in relation to a new sense and perspective of self, via visual implement 500.

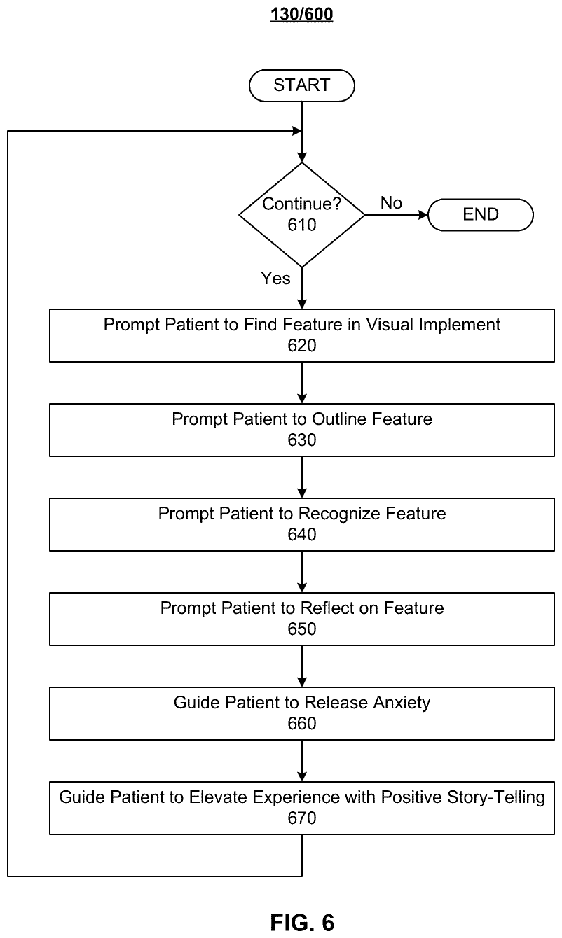

FIG. 6 illustrates an example of subprocess 130, utilizing visual implement 500, according to an embodiment. This embodiment of subprocess 130 may also be referred to herein as process 600, when differentiation from other embodiments is desired. In this embodiment, the clinician may engage the patient using a Find-Outline-Recognize-Reflect-Release-Elevate-by-Story-Telling (FORRREST) strategy, implemented as process 600. The FORRREST strategy is intended to help the clinician safely guide the patient through the integration process, while enabling the patient to release anxiety, elevate the experience, perceive the self in a positive new light, and tell a story. Notably, it is not recommended to use the FORRREST strategy if the patient has experienced a bad trip. In the case of a bad trip, the clinician should preferably utilize the IRON strategy discussed above. It should be understood that, in an alternative embodiment in which process 100 does not utilize a psychedelic substance, the FORRREST strategy can still be used to gauge the patient's mental state, even though there has been no psychedelic experience. In other words, the FORRREST strategy can be used regardless of whether or not there has been a psychedelic experience or the patient has been in a dissociative state.

The FORRREST strategy in process 600 is illustrated as iterative and structured. However, the FORRREST strategy may, instead, be more free-flowing and unstructured, with some of the stages being performed in different orders than illustrated and/or in parallel, and/or one or more of the stages being skipped or omitted. In either case, the clinician or software of system 200 may continue process 600 for as long as it is deemed beneficial (i.e., “Yes” in decision stage 610), and end process 600 once it is deemed to no longer be beneficial (i.e., “No” in decision stage 610). Process 600 may be deemed to no longer be beneficial once no new insights are obtainable.

In the find stage 620, the clinician or the software of system 200 may prompt the patient to find some visual or tactile feature in visual implement 500. The patient may identify an object, a texture, a color, or some other attribute of visual implement 500 as a feature. It should be understood that, while the illustrated example of the visual content of visual implement 500 generally resembles a dog's head, the patient may perceive something different while in the altered state. The clinician should not attempt to correct or otherwise influence what feature the patient finds. Rather, the clinician should allow the patient to explore visual implement 500 and find a feature on the patient's own.

In the outline stage 630, the clinician or the software of system 200 may prompt the patient to outline the feature, found in stage 620. In the event that visual implement 500 is non-electronic, the patient may use a writing instrument (e.g., pen, pencil, highlighter, marker) to outline the feature. However, in an embodiment in which the visual implement 500 is textured and reused over multiple sessions, the patient may use the patient's finger to outline the feature. In the event that visual implement 500 comprises system 200, the patient may use the patient's finger or a stylus to outline the feature in the visual content displayed on touch-panel display 240. In this case, the outline may be overlaid, in real time, as the patient draws it, on the visual content displayed on touch-panel display 240. In addition, the outline may be stored in memory (e.g., main memory 215 and/or secondary memory 220) for future reference.

In the recognize stage 640, the clinician or the software of system 200 may prompt the patient to recognize the feature. In other words, the patient is prompted to describe the feature. This description by the patient may reflect what the patient is currently experiencing or previously experienced during the psychedelic experience. In the event that visual implement 500 comprises system 200, the patient's description may be automatically recorded, via a microphone of system 200, to patient information within memory of system 200.

In the reflect stage 650, the clinician or the software of system 200 may prompt the patient to reflect on the feature, recognized in stage 640. In other words, the patient is prompted to reflect on the feature. This reflection by the patient may relate the feature to the patient's psychedelic or other experience and/or to a new perspective or reflection of the self. In the event that visual implement 500 comprises system 200, the patient's reflection may be automatically recorded, via a microphone of system 200, to patient information within memory of system 200.

In the release stage 660, the clinician or the software of system 200 may guide the patient to release anxiety (e.g., about returning to reality), if any. The clinician may sense such anxiety in the patient's description during stage 640 and/or stage 650.

In the elevate stage 670, the clinician or the software of system 200 may guide the patient to elevate the experience by telling a positive story. For example, the patient may be prompted to find something positive in their description of the feature in stage 640 and/or reflection in stage 650, and prompted to explain why the patient feels positive about that feature. This elevation facilitates the release of anxiety in stage 660, and may influence the patient towards a more positive state of mind, to improve the patient's experience and treatment outcome, and/or to a new perspective or reflection of the self. In the event that visual implement 500 comprises system 200, the patient's story may be automatically recorded, via a microphone of system 200, to patient information within memory of system 200.

### 3.3. Third Example

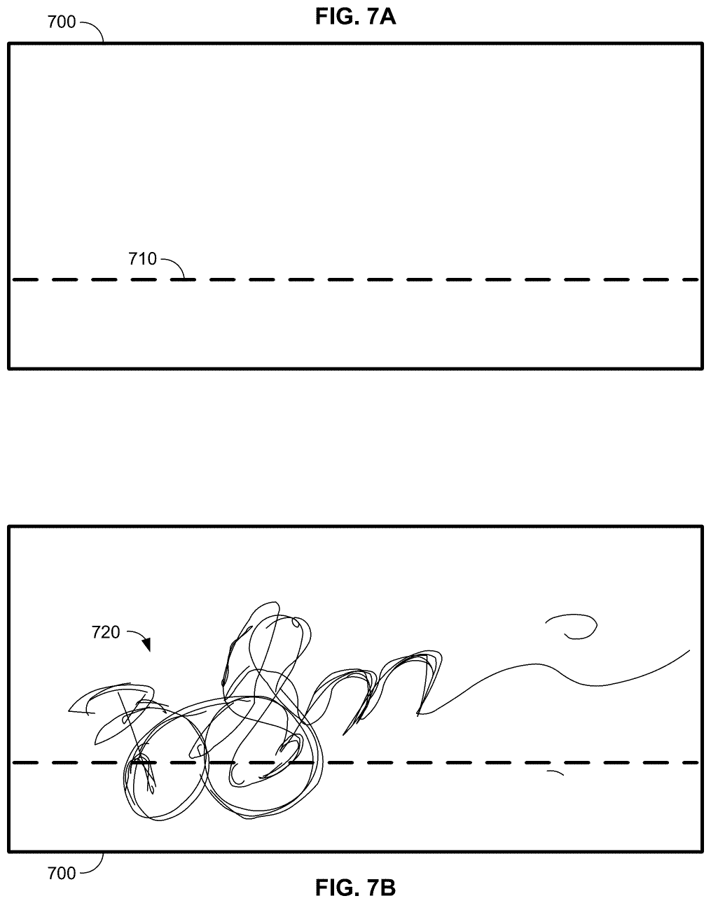

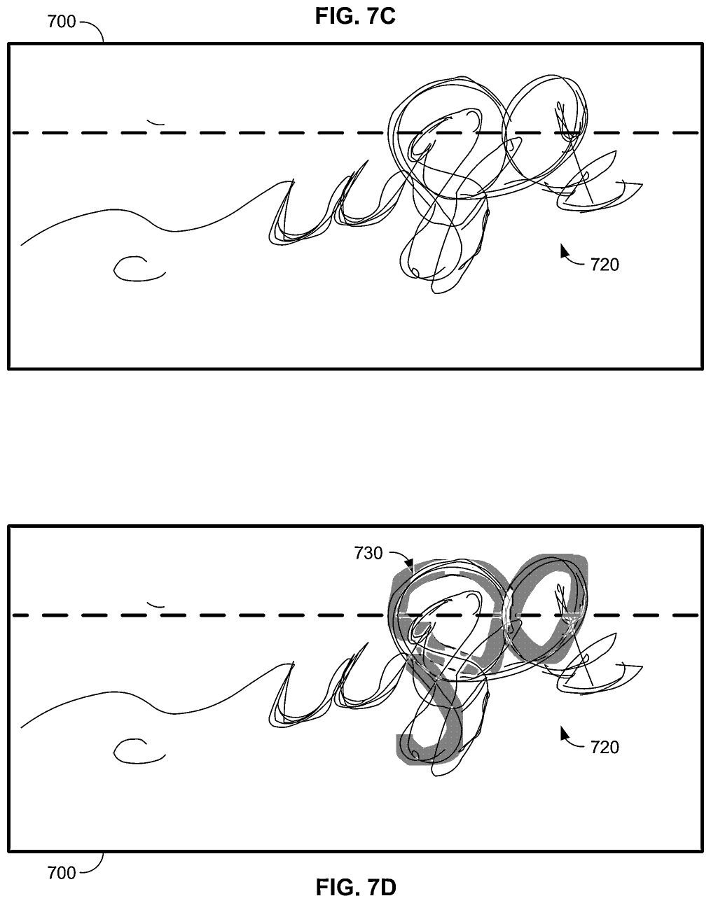

FIGS. 7A-7D illustrate a third example of a visual implement 700 that may be used in subprocess 130, according to an embodiment. At a high level, visual implement 700 comprises a substrate on which the patient may draw in a free-form manner. As illustrated in FIG. 7A, in an embodiment, visual implement 700 comprises a dashed line 710, which may act as a signature line to prompt the patient to draw their signature or other scribble. However, dashed line 710 is not a necessity of any embodiment. In an alternative embodiment, dashed line 710 may be omitted and/or one or more other prompts may be incorporated into visual implement 700. In essence, this third example of visual implement 700 works in the same manner as the first and second examples, except that the patient produces the visual content, and then identifies features in that patient-produced visual content.

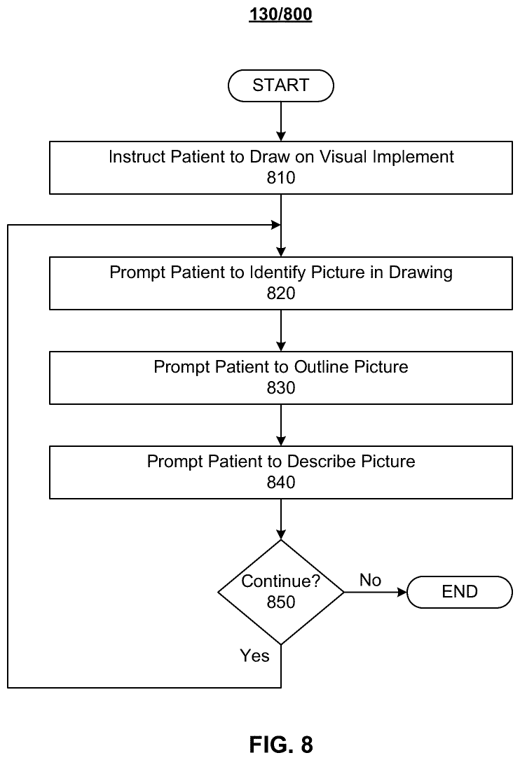

FIG. 8 illustrates an example of subprocess 130, utilizing visual implement 700, according to an embodiment. This embodiment of subprocess 130 may also be referred to herein as process 800, when differentiation from other embodiments is desired. In the same or similar manner as with the first and second examples, visual implement 700, may be shown to the patient, during subprocess 130. Visual implement 700 may be held by the patient or held by the clinician for the patient. Process 800, using visual implement 700 or something similar, may be referred to herein as a Yellow Enhanced Signature (or Scribble) Story (YESS). It should be understood that the YESS strategy, as with any of the other strategies described herein, may be used regardless of whether or not as psychedelic or other substance has been administered to the patient.

In stage 810, the clinician or the software of system 200 may instruct the patient to draw on visual implement 700. The instruction may be to draw something specific, such as a specific object. For example, the patient may be instructed to draw the patient's signature on visual implement 700 (e.g., on dashed line 710). Alternatively, the instruction may be less specific or even vague. For example, the patient may be instructed to simply draw a scribble on visual implement 700. Advantageously, the use of the patient's signature or a scribble does not require significant motor skill or contemplation, and therefore, can be produced by the patient with little to no thought.

Responsive to the instruction, the patient may produce a drawing 720 on visual implement 700. In the event that visual implement 700 is non-electronic, the patient may use a writing instrument (e.g., pen, pencil, highlighter, marker) to draw drawing 720. In the event that visual implement 700 comprises system 200, the patient may use the patient's finger or a stylus to draw drawing 720 on touch-panel display 240. In this case, drawing 720 may be displayed, in real time, as the patient draws it, on touch-panel display 240. In addition, drawing 720 may be stored in memory (e.g., main memory 215 and/or secondary memory 220) for future retrieval and reference.

In any case, the patient's drawing becomes the visual content on visual implement 700. FIG. 7B illustrates visual implement 700 after the patient has drawn a drawing 720 on visual implement 700. In the illustrated example, drawing 720 is the patient's signature or a scribble. While drawing 720 is described as being drawn in subprocess 130, stage 810 can be performed before subprocess 120 (e.g., before, during, or after subprocess 110, or in subprocess 105). In other words, in this alternative embodiment, the patient may produce drawing 720 prior to the psychedelic or other experience and subprocess 130.

Regardless of when drawing 720 is produced, in stage 820, the clinician or the software of system 200 may prompt the patient to identify a picture or other feature in drawing 720. In an embodiment, the clinician may rotate drawing 720 or the clinician or software may instruct the patient to rotate drawing 720, for example, by rotating visual implement 700. FIG. 7C illustrates visual implement 700 after it has been rotated by 180 degrees. It should be understood that the angle of rotation is not important, and may be any arbitrary angle between 0 degrees and 360 degrees (e.g., 45 degrees, 90 degrees, 135 degrees, 180 degrees, 225 degrees, 270 degrees, etc.). It is generally contemplated that the patient would rotate visual implement 700 until the patient identifies some picture in drawing 720. The goal of rotation is to change the patient's perspective of drawing 720, which may be the patient's signature or a scribble, to aid the patient in identifying something new within drawing 720.

In stage 830, once the patient has identified a picture in drawing 720, the clinician or the software of system 200 may prompt the patient to outline the picture. FIG. 7D illustrates visual implement 700 after the patient has drawn an outline 730, representing a picture that the patient has identified. It should be understood that the picture may or may not be discernable to someone other than the patient. In the event that visual implement 700 is non-electronic, the patient may use a writing instrument (e.g., pen, pencil, yellow highlighter, marker) to outline the picture. In the event that visual implement 700 comprises system 200, the patient may use the patient's finger or a stylus to outline (e.g., with yellow highlighting) the picture in the visual content displayed on touch-panel display 240. In this case, outline 730 may be overlaid, in real time, as the patient draws it, on the visual content (e.g., drawing 720, which represents the patient's signature or scribble) displayed on touch-panel display 240. In either case, as discussed elsewhere herein, outline 730 may be a partially transparent overlay and/or may have a color that complements the color of drawing 720. In addition, outline 730 may be stored in memory (e.g., main memory 215 and/or secondary memory 220) for future reference.

In stage 840, the clinician or the software of system 200 may prompt the patient to describe the picture, identified in stage 820. For example, the patient may be prompted to tell a story about the picture. In the event that the drawing is the patient's signature, it should be understood that the patient is being guided to identify, outline, and tell a story about a picture in the patient's signature. Similarly, in the event that the drawing is a scribble, it should be understood that the patient is being guided to identify, outline, and tell a story about a picture in the patient's scribble. The goal with this exercise is for the patient to communicate what the patient is experiencing with reference to a picture found in drawing 720. This provides the clinician or software with a sense of whether the patient is in a good state of mind or bad state of mind, whether or not the patient is aware of the patient's surroundings, and/or the like, and/or provides the patient with the opportunity for integration and expression, relevant to the therapeutic goal of a new perspective or reflection of the self. In the event that visual implement 700 comprises system 200, the patient's description may be automatically recorded, via a microphone of system 200, to patient information within memory of system 200.

Process 800 of identifying, outlining, and describing a picture in drawing 720 may be performed iteratively and/or in a structured manner, or may, instead, be performed in a more free-flowing and/or unstructured manner. In either case, the clinician or software of system 200 may continue process 800 for as long as it is deemed beneficial (i.e., “Yes” in decision stage 850), and end process 800 once it is deemed to no longer be beneficial (i.e., “No” in decision stage 850). Process 800 may be deemed to no longer be beneficial once no new insights are obtainable.

### 4. Baseline Determination

In an embodiment, the baseline status of the patient may be determined prior to the psychedelic experience. For example, the baseline status may be determined by performing one of the FORRREST strategy in process 600, YESS strategy in process 800, and/or IRON strategy in process 400, which may be collectively referred to herein as the FYI processes, in conjunction with a visual implement, in subprocess 105. The baseline status is the patient's observable, physical presentation, demeanor, mood, mindset, and/or affect at the start of a psychedelic-assisted treatment session. Ideally, the clinician should document the patient's baseline status in subprocess 105, for comparison with the patient's altered status in subprocess 120 and/or the patient's egress status in subprocess 130.

In an embodiment, the clinician uses a trigger in advance of performing one of the FYI processes in subprocess 105. The trigger may be any stimulus that is designed to trigger a reaction from the patient and that is preferably not anticipated by the patient. As an example, the trigger may be a delay, caused by the clinician, to the start of the psychedelic-assisted treatment session or other stage of the psychedelic-assisted treatment session. For instance, the clinician may intentionally arrive late to the appointment with the patient, thereby causing the patient to wait for a significant period of time (e.g., thirty minutes). It should be understood that this is simply one example, and the trigger could comprise any other disrupting or otherwise stimulating event. However, in general, the trigger should be mild and reasonable, such that it triggers the patient in a manner that will help the clinician evaluate the patient's baseline status prior to administration of the psychedelic substance, but does not have the potential to damage the patient or the subsequent psychedelic experience. Notably, if the baseline status of the patient is sufficiently concerning to the clinician, the clinician could potentially cancel the session to avoid a bad or dangerous psychedelic experience, and thereafter address an otherwise undetectable need for harm reduction.

Establishment of the baseline status using a trigger may be utilized in any of the disclosed embodiments, and regardless of the psychedelic substance and dose that are used. However, the most benefit may be achieved for intravenous ketamine infusion. This is because intravenous ketamine is used for psychedelic healing interventions that are commonly administered in higher doses (i.e., not as micro-doses) to obtain a full therapeutic effect.

When used with ketamine, the trigger may be designed to minimize harm and maximize safety as much as possible, while preventing an overwhelming or traumatizing experience. A traumatizing experience may involve flashbacks, negative associations with a lack of control, frightening hallucinations, or other factors described in negative ketamine experiences, which are commonly referred to as “k-holes.” K-holes, which are ketamine-induced bad trips, can occur as a result of the extremely frightening, disarming, and uncontrollable features associated with high doses of ketamine (e.g., a therapeutic dose, according to body mass and metabolism). If the patient has a significant amount of anxiety, relative to the patient's first psychedelic experience or any aspect of the clinical setting or other variables, this anxiety can be heightened by the psychoactive effects of ketamine. It can result in a negative experience, which can range from confusing to slightly uncomfortable to very uncomfortable to overwhelming, frightening, and terrorizing. For some patients, this may even result in a worsened perspective and more severe symptoms of the patient's mental health diagnosis, such as hallucination persisting perception disorder (HPPD), or worsened depression, anxiety, substance abuse, and/or suicidality.

Every attempt should be made to avoid the patient experiencing a k-hole. Thus, if the baseline status indicates that the patient may be susceptible to experiencing a k-hole, the clinician may take one or more remedial actions. For example, the clinician may end or postpone the psychedelic-assisted treatment session, reduce the dose of ketamine to be administered, alter the set and setting, and/or the like.

When a k-hole or other bad trip is experienced, there are very few clinical measures in place to assist the patient in terms of the present moment and in terms of immediate reversal of the psychedelic substance. If administered intramuscularly, there is no known way to reverse or stop the psychedelic experience. The patient will be forced to endure any adverse effects for the duration of the psychedelic experience. With intravenous infusion, the patient can be given intravenous benzodiazepines to counter anxiety and its effects. However, this would require communication and translation of the negative experience between the clinician and patient. Unfortunately, k-holes often render the patient without any control whatsoever, leaving the patient without any means to communicate the nature of the experience. Accordingly, the patient may be forced to “ride it out” until the ketamine is metabolized. For intravenous ketamine, the administration of the psychedelic substance and the resulting psychedelic experience typically lasts around sixty to ninety minutes. For intramuscular ketamine, the psychedelic experience will typically begin within two to five minutes, peak between twenty and forty minutes, and last one to three hours. Thus, a k-hole or other negative experience, especially for a patient that is new to psychedelic substances, can flood the patient with fear, anxiety, depression, flashbacks, and more. All of this can result in negative impressions of the psychedelic experience and increased suicidal ideation.

This is the opposite of the intended outcome of psychedelic-assisted therapy or other intervention. The patient may still experience a growth in the glutamate of the prefrontal cortex and other physiological changes within the brain that are associated with improvement and symptom reduction. However, if the patient's first psychedelic experience is negative, the psychedelic experience may result in the opposite of growth and healing, including increased suicidality, which may go unreported. In addition, the patient may terminate the therapy or stop complying with aspects of the therapy.

Thus, if despite the clinician's best efforts, such an experience does occur, the clinician should explore and process the experience using a visual implement (e.g., 300, 500, or 700) with an accompanying one of the FYI processes (e.g., 400, 600, or 800), as described herein, whenever possible. This will give the patient and the clinician the best opportunity to arrive at a therapeutic outcome and to reduce any potential harm. In addition, it will give the patient the opportunity to process, express, and integrate with regard to what was experienced, whether as a flashback, memory, or other feature, or with regard to the memory of trauma itself.

A purpose of disclosed embodiments is to facilitate communication by the patient, for example, before and after the administration of the psychedelic substance. The disclosed embodiments can enable all sentiments and associations with self-harm to be gleaned from the patient, regardless of whether the psychedelic experience was a good trip, a bad trip, or anywhere in between.

In an embodiment, the trigger for establishing the patient's baseline status (e.g., in subprocess 105) is only used with a specific type of patient. For example, the trigger may be used to establish a baseline status for a patient that presents with trauma that may have been experienced in a violent manner, or in a culture which requires systemic and strict training, creates ingrained patterns of behavior and perspective, requires rigid conformance, carries high risk, is very structured with the intent to produce tactical skillsets and teach survival in extreme scenarios, and/or the like. An example of such a patient may be a combat veteran, who has never experienced a psychedelic substance, has never experienced the inherent lack of control in a psychedelic experience, and has numerous symptoms and mental health diagnoses related to trauma (e.g., PTSD, substance use disorder, refractory depression, anxiety, and/or suicidality).

### 5. Embodiments for Visually-Impaired Patients

A patient may be blind or have another visual impairment that prevents them from seeing the visual content on the visual implement. In this case, the visual implement may also be textured, as described elsewhere herein (e.g., with respect to visual implement 500). The same or similar processes may be used with a textured visual implement, as with the visual implement generally. Essentially, the visual features that are described with respect to each of the FYI processes (e.g., 400, 600, 800) may be replaced with tactile features.

For example, the clinician or the software of system 200 may still engage the patient using the IRON strategy, described with respect to process 400. In this case, the feature that is identified in stage 420 will be a tactile feature. The clinician or software may then prompt the patient to relate this tactile feature, outline this tactile feature (e.g., with the patient's finger), and narrate this tactile feature, as before, in stages 430, 440, and 450, respectively.

Similarly, the clinician or the software of system 200 may still engage the patient using the FORRREST strategy, described with respect to process 600. In this case, the feature that is identified in stage 620 will be a tactile feature. The clinician or software may then prompt the patient to outline this tactile feature (e.g., with the patient's finger), recognize this tactile feature, reflect on this tactile feature, release any anxiety, and elevate the experience with a positive story, as before, in stages 630, 640, 650, 660, and 670, respectively.

The clinician or the software of system 200 may also still engage the patient using the YESS strategy, described with respect to process 800. In this case, stage 810 may involve the patient sculpting (e.g., in clay), painting, or otherwise creating a textured implement. Then, in stages 820, 830, and 840, the patient may be prompted to identify a tactile feature in the textured implement, outline the tactile feature, and describe the tactile feature, respectively.

The texture may be produced in any suitable manner. For example, the texture may be produced by brush strokes (e.g., a paintbrush), plaster, glue, molding (e.g., plastic), and the like. It should be understood that the texture may be non-uniform to provide a multitude of discoverable features. The texture may correspond to the visual content, with higher elevations of texture representing features of the visual content that are in the foreground, and lower elevations of texture representing features of the visual content that are in the background. Alternatively, the texture could be unrelated to the visual content, or the visual content may be omitted altogether.

Different materials may be incorporated into the texture to provide a variety of different textures. Examples of such material include, without limitation, plastic, metal, rubber, fabric, feathers, paint, plaster, clay, and/or the like. Thus, the tactile features may be varied over both elevation and material.

### 6. Software Application

As discussed elsewhere herein, the visual implement may be embodied in a software application that provides a graphical user interface via a system 200. This graphical user interface may comprise one or more screens that include the visual content of any of the above examples of visual implement, including visual implements 300, 500, and 700. The screen(s) could also provide the various prompts described with respect to processes 400, 600, and 800. In addition to the visual content and/or implementations of processes 400, 600, and/or 800, the software application may provide further functionality and features to facilitate the psychedelic-assisted treatment.

The software application may be server-based (e.g., in a cloud computing environment). In this case, a clinician or patient may access the software application executing remotely on an external system 255, independently of the particular visual implement that is used during the psychedelic-assisted or other treatment. In other words, the clinician or patient may log in to the software application from any system 200, at any time, to access all or a subset of the functionality and features available through the software application. Alternatively, the software application may be client-based, such that a local copy of the software application executes on a system 200. As yet another alternative, the software application may be distributed such that some modules of the software application execute remotely on an external system 255, and other modules of the software application execute locally on system 200.

Whether server-based, client-based, or distributed, the software application may provide both a clinician version or mode and a patient version or mode. Whether the software application executes in the clinician mode or the patient mode may depend on which version of the software application the user logs in to, or the role of the user who logs in to the software application (e.g., based on a role or set of permissions associated with the authenticated user). For example, each user account may be associated with one of a clinician role, patient role, or other role (e.g., administrator role). When a user associated with the clinician role logs in, the user may be provided with a graphical user interface in the clinician mode. Similarly, when a user associated with the patient role logs in, the user may be provided with a graphical user interface in the patient mode.

In the clinician mode, the graphical user interface may comprise one or more screens and the software application may comprise functionality for inputting and/or viewing clinical information for one or more patients. The clinical information may comprise notes by the clinician for a patient (e.g., observations timestamped with date and time), dosage information for a past, current, or future session with the patient (e.g., dose for the psychedelic substance, procedure for administering the psychedelic substance, warnings, etc.), set and setting information for a past, current, or future session with the patient (e.g., location, patient's preferences, etc.), patient information for the patient (e.g., patient's health record, medical history, session history, etc.), and/or the like. In addition, for each patient that has previously participated in a session of therapeutic (e.g., psychedelic-assisted) treatment, the patient information may comprise all information received from the patient with respect to the visual implement(s) (e.g., 300, 500, and/or 700) and the disclosed FYI process(es) (e.g., 400, 600, and/or 800). This information may comprise audio recordings, responses to prompts, signatures and/or other drawings on the visual implement, other expressions or communications, and/or the like.

In the patient mode, the graphical user interface may comprise one or more screens and the software application may comprise functionality for inputting and/or viewing an electronic record of the patient's treatment. The electronic record may comprise journal entries written by the patient, any or all of the patient information received from the patient with respect to the visual implement(s) and the disclosed process(es), and/or the like. Thus, the patient has access to all of the patient's original creations and expressions, as well as a recorded timeline of the patient's experiential responses, progress, and creative expressions. Some of this information may be private to the patient and not viewable by the clinician. In this case, the software application may provide a mechanism for the clinician to request access to the private information (e.g., via an input of the graphical user interface in the clinician mode) and/or for the patient to grant access to the private information (e.g., via an input of the graphical user interface in the patient mode).

In the clinician mode, the software application may provide access to any of the visual content of any of the visual implements (e.g., 300, 500, and/or 700) and any of the FYI processes (e.g., 400, 600, and/or 800). For example, during a session of therapeutic (e.g., psychedelic-assisted) treatment, the clinician may select the particular visual content and/or FYI process to be used during subprocess 105 and/or subprocess 130, either before or at the start of the respective subprocess. In particular, the graphical user interface may comprise a screen which lists the available visual content and/or FYI processes as selectable menu options, and the clinician may select the desired menu option from the list. In response to the selection, the graphical user interface may retrieve and display the visual content associated with the selection, to facilitate subprocess 105 and/or subprocess 130. The graphical user interface could also supply the prompts associated with the selected FYI process, as displayed text and/or audio.

In an embodiment, the software application may record all of the patient's responses and creative expressions during an FYI process in subprocess 105 and/or subprocess 130. For example, any drawing, writing, or other input by the patient (e.g., via touch-panel display 240) may be saved in memory (e.g., secondary memory 220), and added to the patient information available in the clinician mode and/or patient mode. In addition, system 200 could comprise a microphone (e.g., connected to I/O interface 235), and software application may access the microphone to produce an audio recording of the session. This audio recording may be saved in memory, and added to the patient information available in the clinician mode and/or patient mode. Alternatively or additionally, the audio recording may be automatically transcribed into text, via a speech-to-text engine, and the text may be saved in memory, and added to the patient information available in the clinician mode and/or patient mode. All of the information, including the inputs, audio, and/or text may be synchronized (e.g., via timestamps), so that the entire session can be played back, with the inputs (e.g., drawings) appearing in synchrony with the audio and/or text. Thus, the entirety of subprocess 130 may be automatically recorded for future retrieval, playback, and analysis.

### 7. Example Use Case

A non-limiting use case will now be described, to illustrate how an embodiment of process 100 may be used in one example scenario of a psychedelic-assisted treatment. It should be understood that the described use case is not limiting in any respect, and that no two sessions of process 100 will be the same.

Initially, a clinician may assess the therapeutic goals of a psychedelic-assisted treatment session and the unique traits of the patient in terms of mental health diagnoses, current presentation (e.g., physical and emotional state), familiarity or lack thereof with psychedelic experiences, and/or other patient-specific factors. Following this assessment, the clinician will select one of the disclosed FYI processes (e.g., 400, 600, or 800). Each FYI process may be associated with a particular visual content, or alternatively, the clinician may select the visual content to be used with the selected FYI process. In an embodiment that utilizes the software application, this may involve selecting an input, representing the particular process and/or visual content, in the graphical user interface of the software application. In an embodiment which is not software-based, this may involve the clinician selecting a non-electronic visual and/or textured implement.

In subprocess 105, the clinician engages the patient with a visual implement (e.g., 300, 500, or 700) according to a selected one of the FYI processes (e.g., 400, 600, or 800). For example, the patient may be prompted to identify aspects of the patient's self through the narration of a brief story in relation to feature(s) detected in the visual implement. In the YESS process, the patient will also have the opportunity to create a visual element by creating a signature or scribble. Subprocess 105 assists the clinician in ascertaining a baseline status, representing the patient's current outlook and mental state, prior to the psychedelic experience. As discussed above, the baseline status may be ascertained after a triggering event (e.g., using the YESS process). However, this is not a necessity of any embodiment.

In subprocess 110, the clinician administers a dose of the selected psychedelic substance to the patient within the selected set and setting. The dose and set and setting may be influenced by the baseline status of the patient, established in subprocess 105. For example, the clinician may lower the dose or cancel the session entirely, if the baseline status indicates that the patient is in a worse mental state than expected. In the event that the session is canceled, the clinician may still engage the patient with a visual implement and one of the FYI processes to process the cancellation decision.

In subprocess 120, the clinician monitors the patient as the psychedelic substance takes effect and the patient ingresses into the altered state. The clinician may monitor the patient from the same room or a different room (e.g., via cameras). The clinician may recognize when the patient has egressed from the altered state based on the patient's behavior, the amount of time that has passed since subprocess 110, and/or the patient's vital signs. Once the patient has egressed from the altered state, the clinician will initiate subprocess 130.

In subprocess 130, the clinician again engages the patient with a visual implement (e.g., 300, 500, or 700) according to a selected one of the FYI processes (e.g., 400, 600, or 800). The visual implement and/or selected FYI process may be the same as those selected for subprocess 105. Alternatively, the visual implement and/or selected FYI process may be different from those selected for subprocess 105.

Embodiments which utilize both subprocess 105 and subprocess 130 offer a reflective opportunity or mirror, since the clinician and/or patient will be able to compare the patient's responses to an FYI process before the psychedelic experience to the patient's responses to an FYI process after the psychedelic experience (e.g., in the patient information available through the software application described herein). This may allow the clinician, software, and/or patient to identify any change or positive shift in perception (e.g., related to the interpretation of and recognition of the patient's self) after the psychedelic experience. The clinician or software and the patient can work together to extract insights from the comparisons, and integrate those insights into the patient's future choices, behaviors, and perspectives. The intended result is a decrease in references to negative attributes, a decrease in negative self-perspectives, a reduction in wishes of self-harm, and the start of a self-perspective that highlights self-esteem, self-worth, and self-love. In addition and in contrast to conventional self-reporting, the disclosed processes do not require emphasis on or comparison to past, negative, and maladaptive behaviors and choices.

In addition, the ability to compare the patient's responses before and after the psychedelic experience may also provide vital information about the psychedelic experience for both the clinician and the patient. This information may include the efficacy of the administered dose of psychedelic substance (e.g., whether or not the dose should be adjusted), the efficacy of the set and setting (e.g., whether or not the set and setting should be adjusted), the progress achieved by each psychedelic experience and integration, and/or the like. Other information about the patient's experience may also be collected and documented (e.g., in the clinician's notes and/or patient's journal), such as what the patient found helpful, unhelpful, harmful, or neutral.

### 8. Application to Other Psychotherapies

The disclosed processes and visual implements have primarily been described herein in the context of psychedelic-assisted treatment. However, disclosed embodiments may be applied to other types of psychotherapy, including those which do not involve psychedelic substances or which may not involve any substance at all. In particular, any mental health diagnosis may benefit from psychotherapy utilizing one or more of processes 400, 600, and 800 and/or one or more of visual implements 300, 500, and 700.

This is true regardless of the patient's age (e.g., child, adolescent, or adult). Children and adolescents experience depression, anxiety, attention deficit hyperactivity disorder (ADHD), PTSD, substance use disorders, personality disorders, and other mental health diagnoses, just as adults do. Trauma is a fundamental and causal element related to the symptoms and incidence of these mental health diagnoses. The rise of social media has contributed profoundly to incidents of bullying, racial divisions, cultural disparities, and isolation experienced by school-aged children and adolescents. These individuals are at particularly high risk of suicidality and other self-harm during pivotal periods of physical and psychosocial development. Such individuals are commonly in a vulnerable state of self-criticism and self-judgment, resulting from comparisons set forth by social media, as well as from the influence of the nuclear family, divorce, society, and the constant influence from their peers.

Thus, for instance, process 400 may be used as a stand-alone process (e.g., not as subprocess 130 of process 100) or as part of another psychotherapy process. Process 400 may otherwise be the same. In particular, the clinician or software may guide the patient, using the IRON strategy and a visual implement, such as visual implement 300, 500, or 700.

Similarly, process 600 may be used as a stand-alone process (e.g., not as subprocess 130 of process 100) or as part of another psychotherapy process. Process 600 may otherwise be the same. In particular, the clinician or software may guide the patient, using the FORRREST strategy and a visual implement, such as visual implement 300, 500, or 700.

Similarly, process 700 may be used as a stand-alone process (e.g., not as subprocess 130 of process 100) or as part of another psychotherapy process. Process 700 may otherwise be the same. In particular, the clinician or software may guide the patient, using the YESS strategy and a visual implement, such as visual implement 300, 500, or 700.

It should also be understood that any of the disclosed FYI processes may be used in any setting. In particular, although the FYI processes are primarily described herein as being used in a therapeutic and clinical setting, any of the FYI processes could be employed in any other setting. For example, one or more of the FYI processes may be used in a non-therapeutic setting, such as in a research or diagnostic setting and/or in an informal non-clinical setting.

In addition, particularly in an embodiment in which the visual implement is a system 200, an FYI process may be performed virtually, rather than in person. In this case, system 200 may be connected to the Internet or other communication network and may provide an audio or audiovisual connection between the patient utilizing system 200 and a therapist or other person (e.g., utilizing a counterpart system 200 or other system). This enables the FYI process to be performed as virtual interventions between any two people anywhere in the world.

As one particular example of a stand-alone application of the FYI processes (e.g., without the use of any substance), an FYI process may be used with a patient who has experienced a trauma. The trauma may be a past, recent, or imminent trauma, such as an experience within a war zone, a severe accident, witnessing the loss of a loved one, being the victim of a violent act, and/or the like. In some trauma cases, a patient is so traumatized that the patient is unable to speak. This is especially prevalent in children who have experienced a traumatic event. The child may exhibit a “freeze” or “flee” response, in which the child withdraws or retreats into the safety of the child's own internal world, in order to survive and cope with the trauma, leaving the tools for language behind. Additionally or alternatively, there may be an actual language barrier or inability to directly communicate. When standard language faculties fail, the FYI processes can be used as a means of communication to identify, embrace, and assist with the processing and healing of the trauma in every stage of recovery and across any scenario and population. In essence, the story-telling, artistic, and creative process of YESS, or other FYI process, represents a new language that can be used within the same clinician-patient paradigm, but potentially by someone other than a clinician. As an example, a rescue worker or other first responder, encountering a child who has experienced a traumatic event, may utilize the visual implement (e.g., a non-electronic version or an electronic version as in system 200) within one of the FYI processes to help the child process the trauma.

As another particular example of a stand-alone application of the FYI processes, an FYI process may be used in a research setting. For example, one group of patients may be given a psychedelic or other substance being tested, and another group of patients may be given a placebo as a control. After being given the substance or placebo, each patient may then be evaluated using an FYI process to determine the substance's effectiveness, as well as to determine the effectiveness of the FYI process as a therapeutic intervention.

### 9. Preferred Principles

The implementations of disclosed embodiments may be informed by the following principles, which have guided the inventor. However, it should be understood that these principles are not limiting on any embodiment. Rather, they simply represent the inventor's motivations and intentions, which may aid those seeking to practice the disclosed embodiments.

Psychedelic substances have dissociative properties. Disassociation also occurs involuntarily as a means of surviving severe trauma and repressing that trauma into the folds and shadows of awareness. To provide for safety and predictability, with respect to human error in the administration of a psychedelic substance, the term “harm reduction” should be examined with the utmost scrutiny. Survival and coping strategies are often designed, held, and utilized in a manner that science does not fully understand and which cannot be accessed with full conscious recognition. Even the term “healing” carries the implicit message that the patient is somehow damaged, broken, in need of repair, and imperfect. The nomenclature in the current psychedelic community is that the default mode network (DMN) and the ego is to be bypassed (e.g., “leap-frogged”), and that healing is as simple as skipping a stone over the widely-criticized ego, which is the same mechanism that humans use for self-defense and survival.

Human survival mechanisms, which reside in the default mode network, are in place for a reason: because the patient is still alive. Failing to recognize this truth, and simply labeling a choice, behavior, or response as “maladaptive” or “dysfunctional” disrespects and negates the essence of a human's valiant, unmistakable, undeniable, and instinctive drive to live. No matter what the abuse or degree of suffering or trauma, the fact that a patient is still alive and is engaging with efforts to survive, heal, and change, is a feat in and of itself that is worthy of deep reflection. A reexamination of potential biases and tendencies toward social judgment and piety, with regard to mental health diagnoses and suicide, is called for, with the optimal outcome being an understanding of the strength and character necessary for a survivor to decide to live.

The disclosed embodiments were developed based on the certain and resolute truth that everyone has a story to tell. These disclosed embodiments may access facets of any individual, whether patient or clinician, which comprise the authentic self in any stage, and aim to understand elements of their personal stories, through the expressive and subjective sense of the artistic process and perception. This sense carries no quantification, no “do's” or “don't′s”, and no right or wrong. These stories simply are. And because humans are still here, surviving, thriving, or somewhere in between, those stories need and deserve to be shared, in whatever form in which they might emerge.

An antibiotic can be prescribed to eradicate an infection. A surgical intervention can resolve appendicitis. A quadruple bypass can physiologically revive and save the heart. However, nothing can change, medicate, surgically alter or remove, or deny the facts around what a patient has survived, that the patient has survived it, and that the patient is still present, coping, and dealing with the memories of what, at that time, may have seemed so intolerable that death would have been a welcomed escape. Trauma and its accompanying remnants of depression, anxiety, addiction, and more, cannot be resolved by being removed. Patients who suffer and survive trauma must find the bravest currents within and must show up as their own best medicine. Psychedelic-assisted treatment is unlike any other medical intervention, in that it can assist the soul in showing the physical self—the brain in the body and memories in muscle-how to start over in perceiving the self. Thus, the patient is the medicine. As a result, no two psychedelic experiences are alike. They can never be replicated, and can never be experienced in the same way, even within the same patient.

Therein lies the art of healing, the understanding of psychedelic interventions and experiences, and the courage required to release expectations, surrender self-control, and venture into the unknown and indescribable. The patient becomes the medicine when healthcare providers realize and rise up to meet the fact that, unlike how an antibiotic eradicates infection or a broken bone heals in predictable stages, the wounded soul must forge into the depths of trusting the authentic self. The soul houses the uniqueness and individual expression and experience of trauma. In many cases, this trauma has been inflicted on a level so extreme that recovery and healing become meaningless words and unfamiliar concepts. However, this can be changed if the patient is empowered, understood as the key to be respected, and treated as the medicine. Then, healing can become attainable in a new and lasting way.

Within a safe set and setting and with accountability for harm reduction, psychedelic-assisted treatment can be a portal for becoming. The goal of treatment is to come to a point at which the patient is simply living the truth, in the form of an identity which is no longer defined by the framework of the darkness that was survived, but by the light that has always resided within. The most challenging, and likely most rewarding, shift is a change in the patient's perspective. Psychedelics can provide that perspective, within safe and sacred containers. The unique identity of the soul and the light within holds the answer.

The soul holds the blueprint. The psychedelic substance can unlock the most vulnerable state, in which a surrendering of control and reality, as it has been previously experienced, and the relinquishing of the patient's self as the patient's harshest critic, can meet somewhere in the middle. There, the real meaning of integration can be discovered, and the patient can take the ultimate risk, the biggest leap, to find, recognize, and embrace hope. It is within the glow of the truest presence and expression of hope that self-destruction and self-loathing will fade out.

The utilization of psychedelic substances alone is not sufficient to achieve the goal of a new and positive sense of the self. The disclosed embodiments, including the utilization of psychedelic substances with the visual implement and an FYI process, enable the patient to be the medicine. While the FYI processes are primarily described as being performed during or after the psychedelic experience, they could also be performed before the psychedelic experience (e.g., to establish a baseline status). Regardless of when an FYI process is performed, it supports the patient in a unique approach to the discovery and integration of a newly experienced self, and places the patient on the road to positive change and healing. Along with clinical observations, the FYI processes enable an assessment of the unique and individualized traits of each patient, particularly in relation to self-harm and suicidality.

The FYI processes are designed around a self-narrated story. A request is made to the patient to engage in a creative process, while directly interacting with the clinician. This process reveals descriptive information about the patient's perspective, as it relates to a reflection upon, and ideally a new perception, of the self, while grounding the patient in the present moment.

The FYI processes and visual implements were designed with the intent to facilitate the means of narration with regard to the self. The patient is guided toward a dissolution of the ego and an opportunity to experience a “reboot” of self-identity. The patient may evolve from a perspective, which was originally defined by self-destructive symptoms, maladaptive behaviors, and stigmatized diagnoses, to a perspective that mirrors self-worth, self-love, and self-appreciation.

A goal of the FYI processes is to access and augment the potentially therapeutic benefits of one or more psychedelic experiences by enabling a new, rebooted, or recreated sense of the default mode network. An underlying objective is to address suicidality, which is a devastating, fundamental, and underlying component in the described mental health diagnoses. The inventor believes that the prime motivation for federal permissions to research and legalize psychedelics is to significantly reduce the number of suicides and suicide attempts. Utilization of the disclosed embodiments, especially in a clinical setting, offer a means of support, creative expression, communication, reflection, therapeutic discovery, and insight with regard to the patient's self.

The visual implements and accompanying FYI processes offer the patient a new means of discovering and rebooting the self in a positive light, after encountering the dissociative, regenerative, and healing aspects of the psychedelic experience. The FYI processes offer open-ended “mirrors” that are designed to be self-reflective, insight-based, and creative opportunities for communication. They establish safety, reflection, and integration of the new self by inviting narration and expression with regard to the inner world and perspective of the patient's default mode network, as influenced and/or altered by the psychedelic experience, with the opportunity for positive expression of the self and a positive shift in behaviors and choices. This is in contrast to conventional methods that start with a predefined, symptom-based series of questions and scales that require the patient to compare their current self to past, negatively defined symptoms. The inventor has recognized that such conventional methods may be triggering and problematic, especially for patients at risk of self-harm.

The above description of the disclosed embodiments is provided to enable any person skilled in the art to make or use the invention. Various modifications to these embodiments will be readily apparent to those skilled in the art, and the general principles described herein can be applied to other embodiments without departing from the spirit or scope of the invention. Thus, it is to be understood that the description and drawings presented herein represent a presently preferred embodiment of the invention and are therefore representative of the subject matter which is broadly contemplated by the present invention. It is further understood that the scope of the present invention fully encompasses other embodiments that may become obvious to those skilled in the art and that the scope of the present invention is accordingly not limited.

As used herein, the terms “comprising,” “comprise,” and “comprises” are open-ended. For instance, “A comprises B” means that A may include either: (i) only B; or (ii) B in combination with one or a plurality, and potentially any number, of other components. In contrast, the terms “consisting of,” “consist of,” and “consists of” are closed-ended. For instance, “A consists of B” means that A only includes B with no other component in the same context.

Combinations, described herein, such as “at least one of A, B, or C,” “one or more of A, B, or C,” “at least one of A, B, and C,” “one or more of A, B, and C,” and “A, B, C, or any combination thereof” include any combination of A, B, and/or C, and may include multiples of A, multiples of B, or multiples of C. Specifically, combinations such as “at least one of A, B, or C,” “one or more of A, B, or C,” “at least one of A, B, and C,” “one or more of A, B, and C,” and “A, B, C, or any combination thereof” may be A only, B only, C only, A and B, A and C, B and C, or A and B and C, and any such combination may contain one or more members of its constituents A, B, and/or C. For example, a combination of A and B may comprise one A and multiple B's, multiple A's and one B, or multiple A's and multiple B's.

What is claimed is:

1. A psychedelic-assisted treatment comprising:
administering an effective dose of a psychedelic substance to a patient to induce a psychedelic experience by the patient, wherein the psychedelic experience comprises an altered state of the patient;
monitoring the patient during the psychedelic experience; and
after egress by the patient from the altered state to a normal state, engaging the patient with a visual implement that comprises visual content, wherein engaging the patient comprises prompting the patient to identify, outline, and describe a feature of the visual content.

2. The psychedelic-assisted treatment of claim 1, wherein the visual content comprises at least one object within an environment and a plurality of colors.

3. The psychedelic-assisted treatment of claim 2, wherein the at least one object is an animal.

4. The psychedelic-assisted treatment of claim 2, wherein engaging the patient with the visual implement comprises:
prompting the patient to identify the feature of the visual content;
prompting the patient to relate the feature of the visual content;
prompting the patient to outline the feature of the visual content; and
prompting the patient to narrate the feature of the visual content.

5. The psychedelic-assisted treatment of claim 1, wherein the visual content is textured.

6. The psychedelic-assisted treatment of claim 5, wherein the visual content is a painting.

7. The psychedelic-assisted treatment of claim 5, wherein the feature is a visual or tactile feature of the textured visual content.

8. The psychedelic-assisted treatment of claim 5, wherein engaging the patient with the visual implement comprises:
prompting the patient to find the feature of the visual content;
prompting the patient to outline the feature of the visual content;
prompting the patient to recognize the feature of the visual content;
prompting the patient to reflect on the feature of the visual content; and
guiding the patient to release anxiety and elevate perspective by telling a positive story using the feature of the visual content.

9. The psychedelic-assisted treatment of claim 1, wherein engaging the patient further comprises:
instructing the patient to draw a drawing on the visual implement to produce the visual content;
prompting the patient to identify a picture, as the feature, in the drawing;
prompting the patient to outline the picture; and
prompting the patient to describe the picture.

10. The psychedelic-assisted treatment of claim 9, wherein prompting the patient to identify a picture comprises instructing the patient to rotate the drawing to identify the picture.

11. The psychedelic-assisted treatment of claim 9, wherein instructing the patient to draw a drawing comprises instructing the patient to sign the patient's name.

12. The psychedelic-assisted treatment of claim 9, wherein instructing the patient to draw a drawing comprises instructing the patient to scribble on the visual implement.

13. The psychedelic-assisted treatment of claim 1, wherein the visual implement is a non-electronic substrate, and wherein the visual content is on a surface of the non-electronic substrate that is configured to be drawn upon.

14. The psychedelic-assisted treatment of claim 1, wherein the visual implement is a processing device with an integrated touch-panel display that displays the visual content, receives the outline as an input, and stores the outline in association with the visual content in a memory of the processing device.

15. The psychedelic-assisted treatment of claim 14, wherein the processing device is a tablet computer that comprises:
at least one hardware processor;
a non-transitory computer-readable medium; and
a software application, stored on the non-transitory computer-readable medium, wherein the software application is configured to, when executed by the at least one hardware processor,
receive an input from a user,
in response to the input, display the visual content in a graphical user interface of the software application, and,
after displaying the visual content, receive the outline as the input via the graphical user interface, and store the outline in association with the visual content in the non-transitory computer-readable medium as the memory.

16. The psychedelic-assisted treatment of claim 15, wherein the outline is received as a sequence of strokes, and wherein the psychedelic-assisted treatment further comprises, by the software application:
mapping each stroke in the sequence of strokes to a coordinate system of the visual content and a temporal position with the sequence of strokes, and
in response to a user operation, playing back the sequence of strokes.

17. The psychedelic-assisted treatment of claim 15, further comprising, by the software application:
displaying a list of available visual content in the graphical user interface; and
as the input, receiving a selection of the visual content from the list of available visual content.

18. The psychedelic-assisted treatment of claim 15, wherein prompting the patient comprises one or both of displaying or playing audio of one or more prompts via the graphical user interface.

19. The psychedelic-assisted treatment of claim 1, further comprising, before administering the effective dose of the psychedelic substance to the patient, engaging the patient with the visual implement to establish a baseline status of the patient.

20. The psychedelic-assisted treatment of claim 19, further comprising, before engaging the patient with the visual implement to establish the baseline status of the patient, triggering the patient with at least one stimulus.
